(19) United States
(12) Patent Application Publication
(10) Pub. No.: US 2007/0231271 A1
(43) Pub. Date: Oct. 4, 2007

(54) Topical composition of biocellulose as gel, spray-aerosol, cream and/or aqueous for the treatment of epithelial lesions

(75) Inventor: Silvio Jose de Souza, Parana (BR)

Correspondence Address: MCCORMICK, PAULDING & HUBER LLP, CITY PLACE II, 185 ASYLUM STREET, HARTFORD, CT, 06103, US

(73) Assignee: Wellborn Participacoes Societarias S/C Ltda, Londrina City (BR)

(21) Appl. No.: 11/593,309

(22) Filed: Nov. 6, 2006

(30) Foreign Application Priority Data
Mar. 31, 2006 (BR) PI0601330-9

(51) Int. Cl. A61K 9/12; A61K 31/717

(52) U.S. Cl. 424/45; 514/57

(57) ABSTRACT

TOPICAL COMPOSITION OF BIOCELLULOSE AS GEL, SPRAY-AEROSOL, CREAM AND/OR AQUEOUS FOR THE TREATMENT OF EPITHELIAL LESIONS refers to a new pharmaceutical form that uses bacterial cellulose, also known as biocellulose, and that consists of a practical and effective dressing, easy to be applied, specially its composition as gel, cream, spray-aerosol, or aqueous suspension, for use in the medical field as dressing of skin lesions, and has the advantage of developing a protection barrier over the wound, in addition it offers a greater amount of active agent to the wound due to the fact that it is associated to other substances that contain pharmaceutical properties, and allows easy and homogenous application, providing comfort to the patient during the application.

### INVENTION AND INTEGRAL TECHNOLOGICAL SECTION

This patent, of such privileged invention, reveals a new pharmaceutical form of use and application of bacterial cellulose, also known as biocellulose, as a practical and effective dressing, easy to be applied, and specially presented as a gel, cream, spray-aerosol or aqueous suspension composition.

This object is inserted in the technological section of medical, veterinarian and hygiene science, preferably used in the treatment of the acute phase of wounds, promoting protection, pain relief, cooling, and hydration of the lesion, due to its composition's high concentration of water.

The advantages offered by this object of invention is the treatment of the wound with biocellulose as a gel, spray-aerosol, cream or aqueous suspension, providing the patient maximum comfort upon its application, and among other attributes, prevents dehydration of tissues, accelerates the cicatrisation process and preserves the granulation tissue.

### STATE OF THE TECHNIQUE

Improvements achieved in the treatment of certain types of wounds, for example, burns, varicose ulcers, graft donator areas, and decubitus ulcers, among others, still represents a challenge for professionals who deal with such diseases that cause pain for their patients.

In the state of the technique great quantity of therapeutic methods and dressings to treat such conditions are included.

Recent improvements of science have created new perspectives for skin regeneration and the cicatrisation phenomenon. As a result, conventional techniques have to be constantly adapted to follow new products and materials that are now available.

Classic wound treatments used to protect the lesion, but they did not lead to the cure. New available materials offer protection and also a therapeutic effect in the cure process.

The evolution of products that are applied on the wounds with the aim to cause the isolation of the external environment was slow until the temporary skin substitutes appear, such as the first generation of dressings, originated from bandages and gauzes, elements that has several disadvantages and excellent conditions for the proliferation of germs in the lesion, and that require frequent changes, and that hinders the wound cicatrisation.

A second generation of dressings was the skin substitutes of animal origin, such as porcine skin, amniotic membrane, and corpse skin, and the several problems they present are the low efficacy and high costs, poor adherence to the wound and excellent source of contamination requiring a lot of care.

A third generation of dressings was the synthetic dressings, resulting from special formulations, such as polyurethanes derivates associated or not to collagen and other substances, such as adhesive agents. None of them is biodegradable, and, most of them have a limited field of usage; in addition, they present low level of adherence and, also, require frequent changes.

In a fourth generation of dressings the biocellulose pellicle was developed, revealed by patents BR PI 8404937 and U.S. Pat. No. 4,912,049, which uses it as a temporary skin substitute. Such technology was spread in several countries, and its properties originality and novelty are proved, according to works published by: Fontana et al. Acetobacter Cellulose Pellicle as a Temporary Skin Substitute; Applied Biochemistry and Biotechnology, 1991, 28/25, 253-264; Gatti et Al. Physical characterization of a new biomaterial for wound management; Journal of Materials Science; Material in Medicine, 1994, 5, 190-193, and Pitanguy I et al. Utilizacão de Pelicula de Celulose Como Curativo; Revista Brasileira de Cirurgia, 1988; 78 (5) 317-326.

Biocellulose is produced by bacteria, specifically Acetobacter xylinum, gram-negative bacteria widely present in nature, and its main characteristic is to convert glucose into cellulose in fibril form, and the interlacement by chance of fibrils result in a jelly pellicle. The development of the pellicle's first cellulose layer occurs in the air-liquid interface, in such a way that the subsequent layers are developed above the pre-existing cellulose, which is forced to the low and inside part of the culture medium, according to Borzani & Souza, published in the Biotechnology Letters, volume 17 (11) pages 1271 and 1272, in 1995.

From the bacterial cellulose, it is possible to obtain a paste or aqueous suspension of cellulose microfibrils, according to description in patent BR PI8800781-2 of 1988.

### OBJECT OF INVENTION

This invention shows, by processing the pellicles obtained from bacterial zoogleas that form cellulose microfibrils (biocellulose), the obtainment of biocellulose triturated in thin particles and that have physical properties, interesting to be incorporated in new pharmaceutical form for topical application in wounds such as burns, abrasion, cut, post-surgical surgical wound and ulcers of any etiology.

It is proved that bacterial cellulose is not irritating and innocuous for the wound, and has the capacity to create a protection barrier that remains in the site after the application.

It is common that pharmaceutical and cosmetic formulations contain a glycol that is topically acceptable, and that has humectant properties—such glycols have bacteriostatic action—and a cellulose derivate normally used as thickening agent, and can be represented as hydroxyethylcellulose, hydroxypropylcellulose and carboxymethylcellulose and others.

This invention uses biocellulose as the inert vehicle to add the composition of a new presentation form for the topical treatment of wounds as gel, spray-aerosol, cream or aqueous suspension.

It was observed that this invention, after application and drying, develops a pellicle useful for the treatment, for it develops a mechanical barrier protecting the injured area, in addition, it is an ideal vehicle for the substances of therapeutic action of the formulation.

### DETAILED DESCRIPTION OF THE OBJECT

This invention consists of a practical, effective dressing as gel, cream, aerosol-spray, or aqueous suspension that may be used for any wound that causes loss of the epithelium.

The production process of bacterial cellulose in non-stirred culture medium is comprehended by the fact that the culture medium has a carbon source, preferably fructose, of 0.1 to 15%, a source of nitrogen, preferably found in yeast extract, in a concentration of 0.1 to 10%, and an alcohol, preferably ethylic alcohol, in the ratio of 0.1 to 2%. The culture medium is inoculated with an acetic bacteria suspension, preferably Acetobacter xylinum, in the ratio of 0.1 to 10% of the culture medium volume. The inoculated culture medium is transferred to trays and ferments at a temperature of 20 to 30° C., preferably at 25° C. The biocellulose cover is formed on the surface of the culture medium between 24 and 72 hours, preferably in 48 hours.

The biocellulose covers obtained from the fermentation have impurities and undergo a chemical treatment to eliminate non-cellulosic materials, for example, bacterial cell remains and endotoxin that may cause pyrogenic reactions in the products of pharmaceutical grade. This purification is comprehended by the fact that the obtained membranes are treated in one or more chemical solutions to eliminate non-cellulosic materials, and, preferably, in a Sodium Lauryl Sulfate solution in the concentration of 0.1 to 3%, preferably at 1%, followed by a hydroxide sodium solution at 0.5 to 5%, preferably at 3%, followed by successive rinsing for neutralization of chemical products.

The purified Biocellulose paste is comprehended by the fact that it is triturated, crushed or fractionated added with water in the ratio of one part of biocellulose added with up to 30 parts of water, preferably in the ratio of 1:1, in a blender of high rotation, and triturated for 5 minutes and the excess of water is drained, thus, obtaining the biocellulose base paste. This cellulose paste is sterilized in autoclave at 120° C. for 20 minutes.

The following examples evidence the wide variety of products that can be produced from the bacterial cellulose paste. The examples of formulations illustrated as follows were sterilized before the use by an appropriate method, for example, humid vapor in autoclave or gamma irradiation.

When used for topical applications, biocellulose showed to be effective for the recovering of wounds with the advantage of being incorporated in the composition of active substances that helps and accelerate the cicatrisation process.

### Gel Formulation

Gel is a semi-solid preparation composed by colloidal particles that remain disperse (do not sediment). Biocellulose, such as hydroxyethylcellulose, when dispersed in aqueous medium, donate viscosity to the formulation, forming GEL.

### EXAMPLE 1

Preferably, Natrosol®, or other analogue product, is dispersed in propylene glycol. Sodium chloride along with the biocellulose paste is dissolved in the formulation water that is heated to approximately 60° C. The hydroxyethylcellulose and propylene glycol dispersion is then slowly added to the water. The agitation and heating are kept until the thickening. This composition is here referred as Biocellulose Gel and preferably can have the following composition:

| Composition | % in weight/volume |
|---|---|
| Biocellulose Paste | 1.0–50.0 |
| Hydroxyethylcellulose(Natrosol ®) | 0.5–4.0 |
| Propylene glycol | 20.0–29.0 |
| Water q.s. | 100% |

### EXAMPLE 2

The method of example 1 was repeated, except that 0.9% of sodium chloride was added to make the formulation physiologically acceptable.

### EXAMPLE 3

The method from example 2 was repeated, except that an anti-septic was added, in a quantity of 0.01 to 2.0% in weight/volume of the composition.

### EXAMPLE 4

The method from example 2 was repeated, except that an anti-inflammatory was added in a quantity of up to 2% in weight/volume of the composition, preferably a corticosteroid in the range of 0.5%.

### EXAMPLE 5

The method from example 2 was repeated, except that an antibiotic in the range of 0.5% in weight/volume of the composition was added.

### EXAMPLE 6

The method from example 2 was repeated, except that a combination of an anti-septic and an anti-inflammatory was added.

### Spray-Aerosol Formulation

Concretizations of the invention will be illustrated as follows, referring to the examples of Spray-Aerosol formulation:

### EXAMPLE 1

Topical composition in the form of spray of 1 to 50 parts of weight of the biocellulose suspension in water, in a pressurized recipient.

### EXAMPLE 2

The previous example was repeated and added with 0.9 parts of weight of sodium chloride to make the suspension physiologically acceptable.

### EXAMPLE 3

The previous example was repeated and added with an anti-septic in the Biocellulose suspension.

### EXAMPLE 4

The previous example was repeated and added with an antibiotic in the Biocellulose suspension.

### EXAMPLE 5

The previous example was repeated and added with an anti-inflammatory of topical action in the Biocellulose suspension.

### EXAMPLE 6

The previous example was repeated and added with a topical anesthetic in the Biocellulose suspension.

### EXAMPLE 7

The example 1 was repeated with a combination of one or more agents described in examples 8, 9, 10, 11 and 12.

### EXAMPLE 8

The example 1 was repeated, except that the biocellulose suspension was replaced by a group of solvents with polarity enough to dissolve biocellulose. This formulation is preferably applied on the wound in the form of a spray-aerosol with bacteriostatic agents, and the group of solvents was pressurized. This formulation allowed the biocellulose to develop a pellicle over the wound after evaporation of the solvent system.

### Cream Formulation

Creams are two-phase dispersions non-miscible with themselves, and that aided by an emulsifying form a homogenous system. The addition of the biocellulose paste confers biocellulose microfibrils disperse in the composition to the cream. These microfibrils can be useful in the cicatrisation process, as well as to be the base for controlled liberation of pharmacologically active substances.

### EXAMPLE 1

Melt in water bath at 70-80° C. the agents of the oily phase of the cream, such as fatter, consistency donators, emulsifying and additives. Water and water-soluble components are homogenized and heated until 80° C. and slowly added to the biocellulose paste. Add the water-soluble compounds with the biocellulose suspension to the oily phase slowly, under continuous agitation, until cooling to room temperature. This composition referred to herein as Biocellulose Gel and can have the following composition:

| Composition | % in weight/volume |
|---|---|
| Biocellulose Paste | 1.0–50.0 |
| Oily Phase | 5–20.0 |
| Aqueous Phase | 10–30.0 |
| Water q.s. | 100 |

Aqueous Suspension Formulation

Suspensions are pharmaceutical forms composed of two phases: one liquid and other one solid. Biocellulose microfibrils are insoluble in the liquid phase, but by an increased concentration of biocellulose between 2 to 50% in weight may be suspend or disperse. After the application of this suspension, biocellulose naturally tends to develop a pellicle over the tissue where it was applied.

The concretizations of the invention will be illustrated below referring to the examples of biocellulose Aqueous Suspension formulation:

### EXAMPLE 1

Topical composition in the form of aqueous suspension composed of 1 to 50 parts of weight of Biocellulose in water, in a pressurized recipient.

### EXAMPLE 2

The previous example was repeated and added with 0.9 parts of weight of sodium chloride, pharmaceutical grade to make the suspension physiologically acceptable.

### EXAMPLE 3

The previous example was repeated and added with an anti-septic in the Biocellulose suspension, preferably chlorhexidine was used.

### EXAMPLE 4

The previous example was repeated and added with an antibiotic in the Biocellulose suspension.

### EXAMPLE 5

The previous example was repeated and added with an anti-inflammatory of topical action in the Biocellulose suspension.

### EXAMPLE 6

The previous example was repeated and added with a topical anesthetic in the Biocellulose suspension.

### EXAMPLE 7

The example 1 was repeated with a combination of one or more agents described in examples 2, 3, 4, 5 and 6.

1- TOPICAL COMPOSITION OF BIOCELLULOSE AS GEL, SPRAY-AEROSOL, CREAM AND/OR AQUEOUS FOR THE TREATMENT OF EPITHELIAL LESIONS, characterized by the addition of biocellulose in the composition of dressings presented in gel, spray-aerosol, cream, and aqueous suspension form.

2- TOPICAL COMPOSITION OF BIOCELLULOSE AS GEL, SPRAY-AEROSOL, CREAM AND/OR AQUEOUS FOR THE TREATMENT OF EPITHELIAL LESIONS, characterized by biocellulose being submitted to the purification process through one or more chemical solutions, preferably in a sodium lauryl sulfate solution in the concentration of 0.1 to 3%, preferably 1%, followed by a sodium hydroxide solution of 0.5 to 5%, preferably 3%, followed by successive rinsing.

3- TOPICAL COMPOSITION OF BIOCELLULOSE AS GEL, SPRAY-AEROSOL, CREAM AND/OR AQUEOUS FOR THE TREATMENT OF EPITHELIAL LESIONS, characterized by biocellulose being originated by a triturating process of the biocellulose cover/mantle.

4- TOPICAL COMPOSITION OF BIOCELLULOSE AS GEL, SPRAY-AEROSOL, CREAM AND/OR AQUEOUS FOR THE TREATMENT OF EPITHELIAL LESIONS, as claimed in 3, characterized by the triturating process occurring in the addition of water in the ratio of one part of biocellulose, added up to 30 parts of water, preferably in the ratio of 1:1, and be submitted to drainage process of the water excess and sterilization.

5- TOPICAL COMPOSITION OF BIOCELLULOSE AS GEL, SPRAY-AEROSOL, CREAM AND/OR AQUEOUS FOR THE TREATMENT OF EPITHELIAL LESIONS, as claimed in 1, characterized by the aqueous gel form being constituted of biocellulose, water and emollient agents.

6- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 5, characterized by the fact that it includes as emollient agent a derivate of a glycol.

7- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 5, characterized by the fact that it includes up to 1% of sodium chloride in the composition.

8- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 5, characterized by the fact that a compound with therapeutic function, such as an anti-septic, is added.

9- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 5, characterized by the fact that a compound with therapeutic function, such as an antibiotic, is added.

10- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 5, characterized by the fact that a compound with therapeutic function for the wound cicatrisation, such as an anti-inflammatory, is added.

11- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 5, characterized by the fact that a compound with therapeutic function for the wound cicatrisation, such as a topical anesthetic, is added.

12- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 5, characterized by the fact that a compound with therapeutic function for the wound cicatrisation, or a combination of more agents with therapeutic functions, is added.

13- TOPICAL COMPOSITION OF BIOCELLULOSE AS GEL, SPRAY-AEROSOL, CREAM AND/OR AQUEOUS FOR THE TREATMENT OF EPITHELIAL LESIONS, as claimed in 1, characterized by being presented as spray-aerosol, stored in a pressurized recipient, and for having biocellulose in aqueous suspension, preferably dissolved in a solvents system, in its composition.

14- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that biocellulose is dissolved in a solvent system and applied over the wound, and the solvent evaporate, and biocellulose result in form of protection pellicle over the wound.

15- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that it includes a lithium salt to dissolve biocellulose in a solvents system.

16- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that it includes a humectant, preferably a glycol, in the composition.

17- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that a compound with therapeutic function for the wound cicatrisation, such as an anti-septic, is added.

18- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that a compound with therapeutic function for the wound cicatrisation, such as an antibiotic, is added.

19- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that a compound with therapeutic function for the wound cicatrisation, such as an anti-inflammatory, is added.

20- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 13, characterized by the fact that a compound with therapeutic function for the wound cicatrisation, such as a topical anesthetic, is added.

21- Composition, according to claims 13 to 20, characterized by the fact that a compound or a combination of more agents with therapeutic function for the wound cicatrisation is added.

22- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 1, characterized by being presented as a cream and includes biocellulose in its composition.

23- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 22, characterized by the fact that it includes an anti-septic in its composition.

24- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 22, characterized by the fact that it includes an antibiotic in its composition.

25- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 22, characterized by the fact that it includes an anti-inflammatory in its composition.

26- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 22, characterized by the fact that it includes a topical anesthetic in its composition.

27- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 22, characterized by the fact that a compound or a combination of more agents with therapeutic function for the wound cicatrisation is added.

28- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to what is claimed in 1, characterized for being presented as aqueous suspension and by the fact that it includes biocellulose in its composition.

29- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes a humectant agent, more specifically a glycol, in its composition.

30- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes up to 1% of sodium chloride in its composition.

31- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes an anti-septic in its composition.

32- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes an antibiotic in its composition.

33- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes an anti-inflammatory in its composition.

34- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that it includes a topical anesthetic in its composition.

35- TOPICAL COMPOSITION OF BIOCELLULOSE AS GEL, SPRAY-AEROSOL, CREAM AND/OR AQUEOUS FOR THE TREATMENT OF EPITHELIAL LESIONS, according to claim 28, characterized by the fact that a compound, or a combination of more agents, with therapeutic function for the wound cicatrisation is added.
